(19) United States
(12) Patent Application Publication
(10) Pub. No.: US 2026/0115402 A1
(43) Pub. Date: Apr. 30, 2026

(54) HEAT AND MOISTURE EXCHANGE MEDICAL DEVICES

(71) Applicant: Hansa Medical Products, Inc., Carmel, IN (US)

(72) Inventor: Eric D. Blom, Carmel, IN (US)

(21) Appl. No.: 18/912,987

(22) Filed: Oct. 11, 2024

Related U.S. Application Data
(63) Continuation-in-part of application No. PCT/US2023/065672, filed on Apr. 12, 2023.
(60) Provisional application No. 63/329,987, filed on Apr. 12, 2022.
(60) Provisional application No. 63/621,799, filed on Jan. 17, 2024.

(51) Int. Cl. A61M 16/10; A61M 16/04; A61M 16/20

(52) U.S. Cl. CPC A61M 16/1045; A61M 16/0465; A61M 16/201

(57) ABSTRACT

Illustrative embodiments of heat and moisture exchange (HME) medical devices, as well as related methods, are disclosed. According to one aspect, an HME medical device may comprise an HME material configured to absorb heat and moisture from exhaled air and to return the heat and moisture to inhaled air. The HME medical device may also comprise a base including at least one wall defining a passageway for the exhaled and inhaled air, a lid that retains the HME material, and a tether connecting the lid to the base. The lid of the HME medical device may be movable between (i) a first position in which the lid is engaged with the base and the HME material is positioned in the passageway and (ii) a second position in which the lid is disengaged from the base and the HME material is not positioned in the passageway.

### CROSS-REFERENCE TO RELATED APPLICATIONS

This application is a continuation-in-part of International Application No. PCT/US2023/065672, filed Apr. 12, 2023, which claims the benefit of U.S. Provisional Ser. No. 63/329,987 , filed Apr. 12, 2022, and claims the benefit of U.S. Provisional Ser. No. 63/621,799 , filed Jan. 17, 2024. The entire disclosure of each of the foregoing applications is incorporated herein by reference.

### TECHNICAL FIELD

The present disclosure relates to heat and moisture exchange (“HME”) medical devices.

### BACKGROUND

As illustrated in FIGS. 1A and 1B, total laryngectomy (i.e., surgical removal of the voice box) results in anatomical disconnection of the upper and lower airway and a permanent breathing opening in the neck. Prior to laryngectomy (FIG. 1A), normal breathing primarily occurs via the nose. As inspired air passes through the nose, it is warmed, humidified, and (to a limited degree) filtered prior to entering the lungs. On expiration, the nose retains most of the moisture in the exhaled breath. After laryngectomy (FIG. 1B), however, when breathing only occurs via the opening in the neck and not the nose, these warming, humidifying, and filtering functions are missing. As a direct result, the lower airway tends to become dried and irritated, as shown in FIG. 2A. More frequent, and often excessive, mucus production and coughing (through the neck opening) become the new normal.

As illustrated in the FIG. 2B, an HME medical device can alleviate these issues somewhat by performing warming, humidifying, and/or filtering functions when worn in front of the tracheostoma neck opening of a laryngectomy patient. In this way, HME medical devices act as a sort of “artificial nose.” Rolled corrugated paper and/or foam material form the basis of contemporary HME medical devices. As warmed and humidified (moist) exhaled air passes through the HME material (the foam or paper), heat and moisture are deposited in HME material, only to be picked up and returned back to the lower airway by the inhaled airstream.

Prior art HME medical devices typically include two primary components: (1) the HME material (e.g., foam or rolled, corrugated paper) and (2) a cassette, often made of plastic, for holding the HME material. When a patient uses a breathing tube (e.g., an endotracheal or tracheostomy tube), the HME medical device can be positioned and held in the airstream by inserting the HME medical device in-line (e.g., between two sections of a breathing tube or on a proximal end of a breathing tube). For patients not using a breathing tube, the HME medical device is typically positioned and held directly in front of the airway opening into the body by a baseplate that is adhered to the peristomal skin that surrounds the opening in the neck leading into the trachea, as shown and described, for example, in U.S. Pat. No. 11,433,204, the entire disclosure of which is incorporated by reference herein. This type of baseplate includes a cylindrical sleeve for receiving the cassette that holds the HME material.

In many prior art HME medical devices, such as that shown and described in U.S. Pat. No. 11,433,204, the cassette has a circumferential rib on its outer surface that is received in a corresponding groove on the inner surface of the cylindrical sleeve of the baseplate. Removal of the cassette from the baseplate, which must be done each time the user needs to cough, requires the use of both hands (one hand to hold the baseplate against the skin, while the other hand pulls the cassette away from the baseplate). This operation must be done quickly, to avoid fouling the HME material with mucus (which occurs if the HME medical device is not removed in time before coughing), but also carefully, to avoid loosening the adhesive attachment of the baseplate to the skin. Additionally, even if the cassette is successfully removed from the baseplate in a timely manner, the patient is left holding the HME medical device in his or her hand (or setting it somewhere that it could be soiled) during the coughing episode.

### SUMMARY

According to one aspect of the present disclosure, an HME medical device may comprise an HME material configured to absorb heat and moisture from exhaled air and to return the heat and moisture to inhaled air. The HME medical device may also comprise a base including at least one wall defining a passageway for the exhaled and inhaled air, a lid that retains the HME material, and a tether connecting the lid to the base. The lid of the HME medical device may be movable between (i) a first position in which the lid is engaged with the base and the HME material is positioned in the passageway and (ii) a second position in which the lid is disengaged from the base and the HME material is not positioned in the passageway.

### BRIEF DESCRIPTION OF THE DRAWINGS

The detailed description particularly refers to the following figures, in which:

FIG. 1A illustrates patient anatomy before total laryngectomy;

FIG. 1B illustrates patient anatomy after total laryngectomy;

FIG. 2A illustrates humidity and temperature of inhaled and exhaled air through a tracheostomy without an HME medical device;

FIG. 2B illustrates humidity and temperature of inhaled and exhaled air through a tracheostomy with an HME medical device;

FIG. 3 illustrates a first side view of one illustrative embodiment of an HME medical device according to the present disclosure;

FIG. 4 illustrates a second side view of the HME medical device of FIG. 3;

FIG. 5 illustrates a third side view of the HME medical device of FIG. 3;

FIG. 6 illustrates another view of the HME medical device of FIG. 3, from a similar perspective as FIG. 3, but with a flap valve of the HME medical device being pressed closed by a patient;

FIG. 7 illustrates a top view of the HME medical device of FIG. 3, with its lid disengaged from its base while the lid retains the HME material;

FIG. 8 illustrates another view of the HME medical device of FIG. 3, from a similar perspective as FIG. 7, but with the HME material removed from the lid;

FIG. 9 illustrates a graph of test data relating to the HME medical device of FIG. 3, showing the compressive force (in newtons) sufficient to close the flap valve in a manner similar to FIG. 6; and

FIG. 10 illustrates another graph of test data relating to the HME medical device of FIG. 3, showing the tensile force (in newtons) sufficient to disengage the lid from the base.

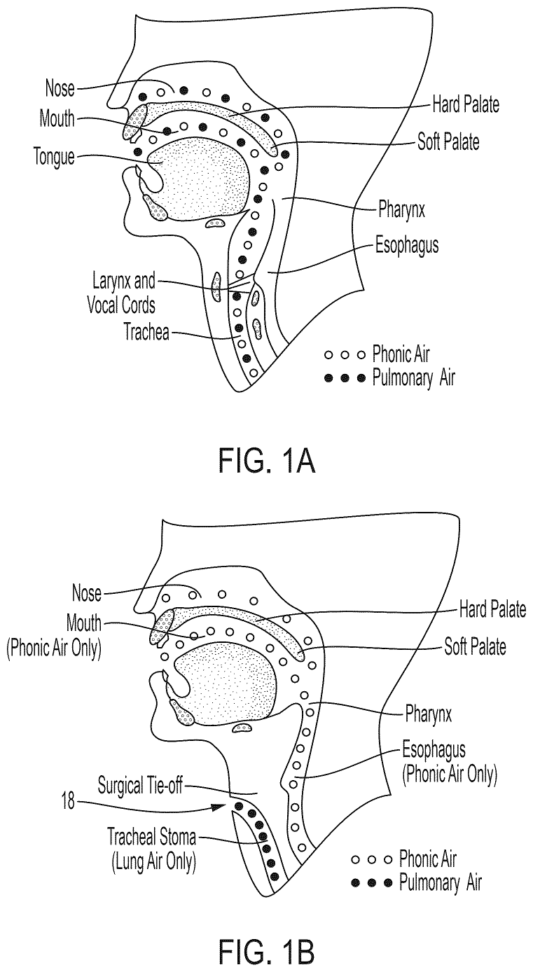

### DETAILED DESCRIPTION OF THE DRAWINGS

While the concepts of the present disclosure are susceptible to various modifications and alternative forms, specific exemplary embodiments thereof have been illustrated by way of example in the drawings and will herein be described in detail. It should be understood, however, that there is no intent to limit the concepts of the present disclosure to the particular forms disclosed, but on the contrary, the intention is to cover all modifications, equivalents, and alternatives falling within the spirit and scope of the invention.

The present disclosure provides improved HME medical devices for post-laryngectomy/tracheostomy patients that are easier, more successful, and more convenient to use. Additionally, the presently disclosed HME devices are economical to produce due to their design.

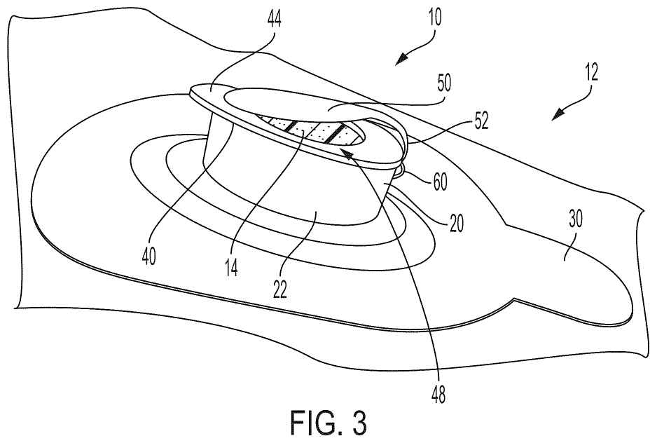

As shown, by way of example, in the illustrative embodiment of FIGS. 3-8, the presently disclosed HME medical devices 10 utilize a body 12 to support an HME material 14. The body 12 of the HME medical device 10 generally includes a base 20, a lid 40, and a tether 60 (each of which are described in more detail below). The body 12 (including the base 20, the lid 40, and/or the tether 60) may be formed of non-porous and non-breathable material(s), such as flexible, thermoplastic material(s). The body 12 may be integrally formed from a molded polymer, such as a polyvinyl chloride (PVC). Alternatively, the lid 40 and the tether 60 may be integrally formed from a molded polymer, such as PVC, while the base 20 is formed as a distinct component (e.g., of molded PVC). As another alternative, the base 20 and the tether 60 may be integrally formed from a molded polymer, such as PVC, while the lid 40 is formed as a distinct component (e.g., of molded PVC). In still other alternative embodiments, the base 20, the lid 40, and the tether 60 may each be formed as distinct components.

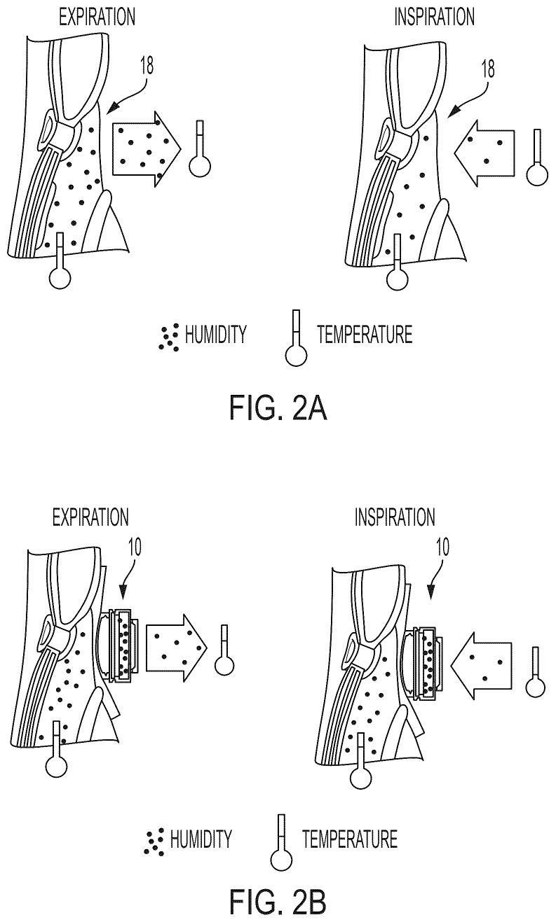

In contrast to the body 12, the HME material 14 that is supported by the body 12 is porous, breathable, and compressible. As suggested by FIG. 2B (discussed above), the HME material 14 is configured to absorb heat and moisture from exhaled air that passes through the HME material 14 and to return the heat and moisture to inhaled air that passes through the HME material 14. The HME material 14 may comprise a foam and/or rolled, corrugated paper. In the illustrative embodiment shown in FIGS. 3-8, the HME material 14 is disk-shaped. It will be appreciated that, while the illustrative embodiment utilizes foam as the HME material, other embodiments of the presently disclosed HME medical devices may use any other suitable HME material.

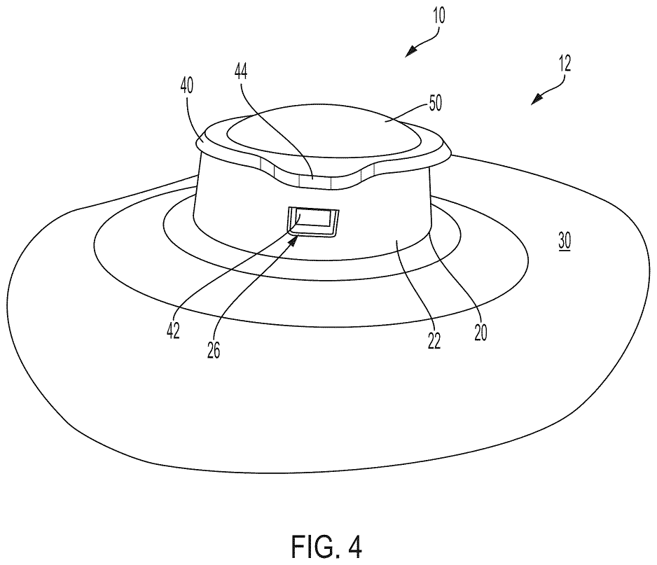

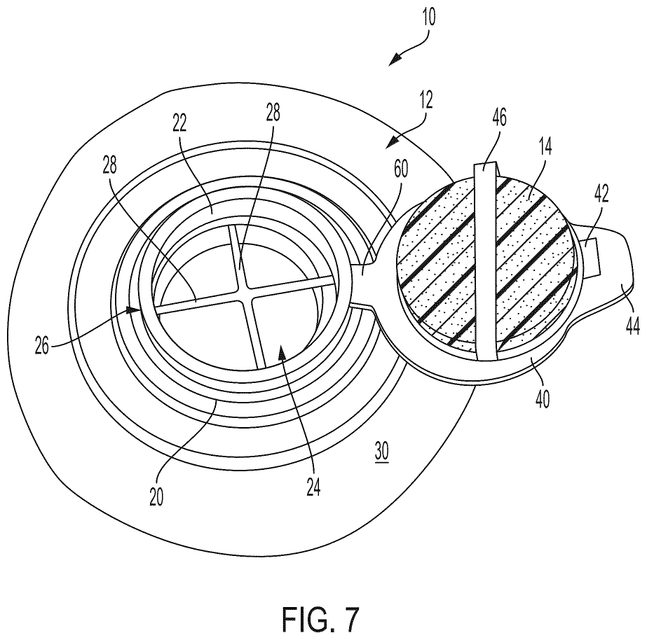

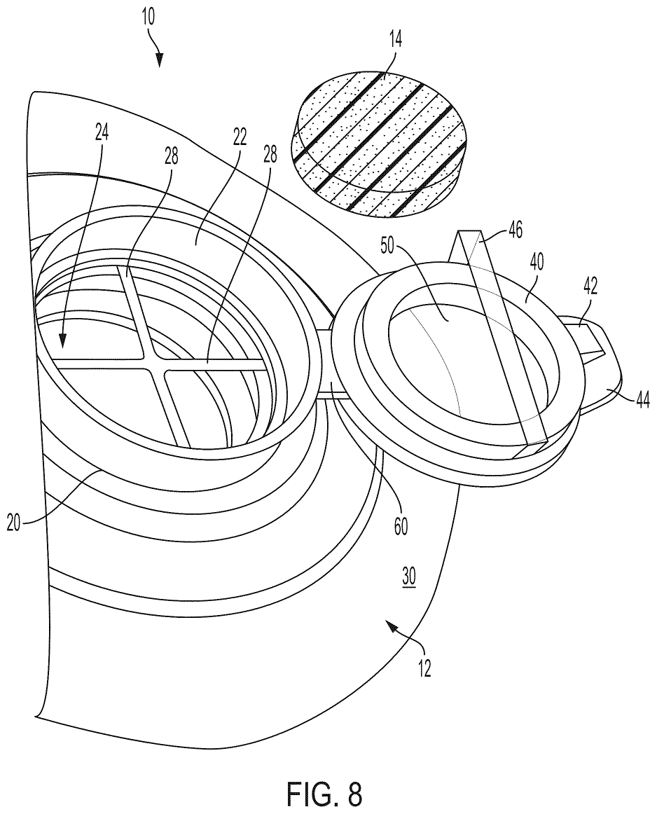

The base 20 of the body 12 includes at least one wall 22 that defines a passageway 24 for the exhaled and inhaled air. In the illustrative embodiment shown in FIGS. 3-8, the wall 22 is cylindrical. As best seen in FIG. 4, the cylindrical wall 22 is formed with an opening 26. As further described below, this opening 26 serves as a mating feature for releasably securing the lid 40 relative to the base 20 during use. The cylindrical wall 22 may be otherwise devoid of openings, such that air cannot escape the passageway 24 through the wall 22. As best seen in FIGS. 7 and 8, the base 20 of the illustrative embodiment also includes a pair of cross-bars 28 to as a precaution against the HME material 14 entering the tracheostoma 18 (if the HME material 14 were to separate from the lid 40). The cross-bars 28 are orthogonal and each extend across a diameter of the cylindrical wall 22. In other embodiments, other numbers of cross-bars 28, or even other features (e.g., tabs) may be used for this purpose.

The base 20 of the illustrative embodiment further includes an annular, flexible, resilient skirt 30 projecting outwardly from the cylindrical wall 22 (it will be appreciated that the skirt 30 could have other shapes, such as other polygons). The skirt 30 is configured to be affixed to a patient's skin adjacent a tracheostoma, such as around the tracheostoma neck opening 18. One side of the skirt 30 may be coated with a suitable adhesive so that the skirt 30 may be removably adhered to the peristomal skin of the patient. In other embodiments (which do not utilize skirt 30), the HME medical device 10 may further comprise a respiratory device (not shown) defining an airway and the base 20 of the body 12 may be configured to open into the airway. In such embodiments, the respiratory device may comprise a laryngectomy or tracheostomy tube, by way of example.

The lid 40 of the body 12 supports and retains the HME material 14. The lid 40 can be selectively engaged with or disengaged from the base 20. As shown in FIGS. 3-6, the lid 40 has an engaged position in which the lid 40 is engaged with the base 20 and the HME material 14 is positioned in the passageway 24. As shown in FIGS. 7-8, the lid 40 also has at least one disengaged position in which the lid 40 is disengaged from the base 20 and the HME material 14 is not positioned in the passageway 24. The patient may selectively move the lid 40 between the engaged position (to place the HME material 14 in the path of exhaled and inhaled air) and the disengaged position (to remove the HME material 14 from the airway, for instance, when the patient needs to cough). It is contemplated that the lid 40 may be embodied as a HME cartridge or cassette that supports and retains the HME material 14 (like the cassette described in U.S. Pat. No. 11,433,204).

In the illustrative embodiment, the lid 40 is circular and has a similar diameter to the cylindrical wall 22 of the base 20. The lid 40 is designed to engage with the base 20 via a snap fit when the lid 40 is in its engaged position. To do so, the lid 40 includes a mating feature that is configured to engage with a mating feature of the base 20 when the lid 40 is in its engaged position. While any number of corresponding mating features may be used, the illustrative embodiment includes a tab 42 on the lid 40 that is received in the opening 26 formed in the wall 22 of the base 20 (described above). It is contemplated that, whichever mating features are used, the lid 40 will be relatively easy for the patient to disengage from the base 20. For instance, in the illustrative embodiment, the patient can disengage the lid 40 from the base 20 with a single hand. To aid in this operation, the lid 40 includes a handle 44 that extends outwardly beyond the wall 22 of the base 20 when the lid is in the engaged position. The patient can grip and/or pull the handle 44 with one or more of their fingers to disengage the lid 40 from the base 20 with relative ease.

Testing of prior art HME medical devices revealed that tensile forces of greater than 9 newtons were required to remove the cassettes (carrying the HME material) from their baseplates. In particular, a 9.1 N peak removal force was required for an Atos Medical HME medical device, while an 11.5 N peak removal force was required for an InHealth Technologies HME medical device. By contrast, the illustrative embodiment of the presently disclosed HME medical device 10 required only about 0.7 N of peak removal force to disengage the lid 40 from the base 20, as shown in the test results presented in FIG. 10. It is contemplated that, in other embodiments of the presently disclosed HME medical device 10, a force of less than 1 N (i.e., between 0-1 N), or less than 3 N (i.e., between 0-3 N), or less than 5 N (i.e., between 0-5 N), or less than 9 N (i.e., between 0-9 N) may be sufficient to disengage the lid 40 from the base 20.

Testing of the presently disclosed HME medical device 10 also revealed that air pressure of about 11 centimeters of water in the passageway 24 (such as might be experienced during a patient's cough) was sufficient to disengage the lid 40 from the base 20. Specifically, air pressures of 8.8, 11.9, and 12.2 centimeters of water in the passageway 24 were found to be sufficient across three tests of the HME medical device 10. By contrast, tests using air pressures up to 120 centimeters of water were not able to dislodge the HME cassettes from their baseplates in the prior art HME medical devices (from Atos Medical and InHealth Technologies). It is contemplated that, in other embodiments of the presently disclosed HME medical device 10, air pressures of less than 15 centimeters of water (i.e., between 0-15 cm H₂O), or less than 50 centimeters of water (i.e., between 0-50 cm H₂O), or less than 100 centimeters of water (i.e., between 0-100 cm H₂O) may be sufficient to disengage the lid 40 from the base 20. In other embodiments, air pressures between 8 and 13 centimeters of water may be sufficient to disengage the lid 40 from the base 20.

Additionally, the lid 40 includes one or more features to assist in retaining HME material 14 adjacent the lid 40. Preferably, these features temporarily retain the HME material 14 relative to the lid 40, such that the HME material 14 is removable from the lid 40 (see FIG. 8) and can be replaced with another, fresh HME (e.g., if the HME material 14 is fouled). Although removable HME material 14 is preferred, it is also contemplated that the HME material 14 may be permanently affixed to the lid 14 in some embodiments. In either case, the entire HME medical device 10, including the body 12 and the HME material 14, may be provided together in a single sterile package, or in multiple, separate sterile packages.

In the illustrative embodiment, the lid 40 includes a single cross-bar 46 that retains the HME material 14 adjacent the lid 40 (unless and until the patient intentionally removes the HME material 14 for replacement), as best seen in FIGS. 7 and 8. In other embodiments, any number of cross-bars 46 (e.g., a plurality of cross-bars 46) could be used to retain the HME material 14 adjacent the lid 40. Each of the cross-bars 46 may extend from one side of the lid 40 to an opposite side of the lid 40. It will be appreciated that many other features may additionally or alternatively be used to retain the HME material 14 adjacent the lid 40. For instance, the lid 40 may include one or more tabs (e.g., a plurality of tabs) that each extend inwardly from a wall of the lid 40 to retain the HME material 14 adjacent the lid 40. In such embodiments, the patient may remove/insert the HME material 14 through a center space between the inwardly extending tabs. In some embodiments, an interior surface of the lid 40 may be coated with a suitable adhesive to retain the HME material 14 adjacent the lid 40. In some embodiments, the lid 40 may include one or more posts extending down from the interior surface of the lid 40 and the HME material 14 may be retained adjacent the lid 40 by inserting the post(s) through the HME material 14.

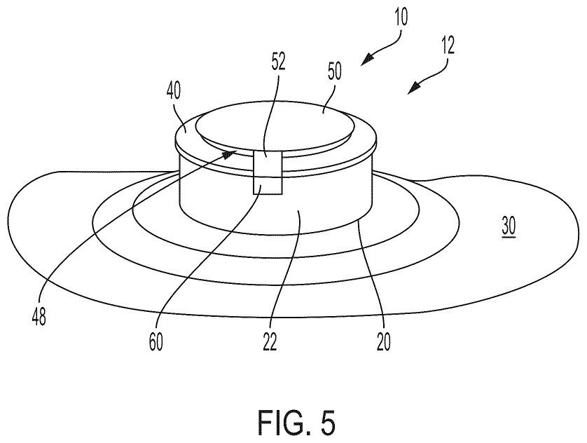

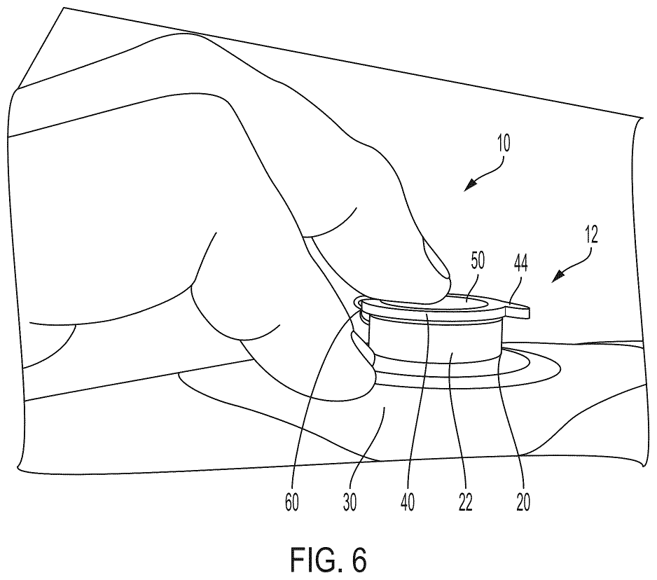

As shown in FIGS. 3 and 5, the lid 40 may be formed to include one or more openings 48 that allow exhaled and inhaled air to pass through the lid 40 (and, thus, through the HME material 16) when the lid 40 is in the engaged position. In the illustrative embodiment, the lid 40 includes a single, central opening 48 in the lid 40 for this purpose. The lid 40 of the illustrative embodiment further includes a flap valve 50 that is configured to selectively seal the opening 48 (e.g., to assist the patient in speaking). The flap valve 50 is moveable between an open position, in which at least a portion of the flap valve 50 is spaced from the opening 48, as shown in FIGS. 3-5, and a closed position, in which the flap valve 50 is seated on the lid 40 to cover the opening 48, as shown in FIG. 6. In the illustrative embodiment, the flap valve 50 is biased toward the open position (shown in FIGS. 3-5) by a small strip 52 of resilient material, which may be integrally formed with the rest of the body 12 in some embodiments.

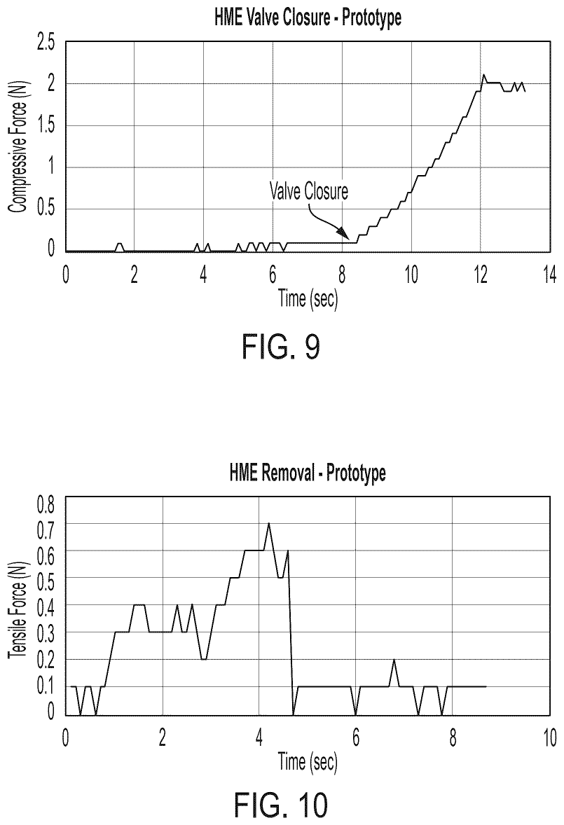

As suggested by FIG. 6, the flap valve 50 may be moved from the open position to the closed position by a patient pressing a finger on an outer surface 54 of the flap valve 50. Testing of the illustrative embodiment of the presently disclosed HME medical device 10 demonstrated that only about 0.1 N of peak compressive force was needed to seat the flap valve 50 against the lid 40 to selectively seal the opening 48, as shown in the test results presented in FIG. 9. By contrast, testing of prior art HME medical devices revealed that significantly greater compressive forces were required: specifically, a 1.8 N peak valve force for the Atos Medical HME medical device, and an 5.7 N peak valve force for the InHealth Technologies HME medical device. It is contemplated that, in other embodiments of the presently disclosed HME medical device 10, a force of less than 0.5 N (i.e., between 0-0.5 N), or less than 1 N (i.e., between 0-1 N), or less than 1.5 N (i.e., between 0-1.5 N), or less than 1.7 N (i.e., between 0-1.7 N) may be sufficient to seal the opening 48 with the flap valve 50.

The tether 60 of the body 12 connects the lid 40 to the base 20. As such, even when the lid 40 is disengaged from the base 20, the lid 40 may be maintained within a predetermined distance of the base 20. In the illustrative embodiment, the tether 60 is connected to the lid 40 opposite the tab 42 and connected to the base 20 opposite the opening 26. Since the tether 60 has a relatively small thickness, it functions as a living hinge and, thus, provides a hinged connection between the lid 40 and the base 20. When the patient needs to move the HME material 14 out of the airway (e.g., due to an oncoming cough), it is relatively easy for the patient to pull on the handle 44 of the lid 40 to disengage the lid 40 from the base 20. The HME material 14 is then out of the airway but, due to the tether 60, need not be held or set down by the wearer. Instead, the lid 40 may hang from base 20 via the connection provided by the tether 60 during a coughing episode (without becoming soiled or lost).

In the illustrative embodiment shown in FIGS. 3-8, the tether 60 is integrally formed with the base 20 and the lid 40. It is also contemplated that, in other embodiments, the tether 60 may be formed distinctly from the base 20 and/or the lid 40. In such embodiments, the tether 60 may be separable from the base 20 and/or the lid 40. In these alternative embodiments, the tether 60 may be connected to the base 20 and/or the lid 40 using any number of suitable methods. For instance, an adhesive may be used to connect the tether 60 to the base 20 and/or the lid 40. Alternatively, a mechanical connection may be established between the tether 60 and either or both of the base 20 and the lid 40. By way of example, either end of the tether 60 could include a nub or protuberance that could be inserted into a corresponding opening formed in the base 20 and/or the lid 40. It will be appreciated by those skilled in the art that any number of known connections could be used to connect the tether 60 to the base 20 and/or the lid 40, as needed.

While the disclosure has been illustrated and described in detail in the drawings and foregoing description, such illustration and description is to be considered as exemplary and not restrictive in character, it being understood that only illustrative embodiments have been illustrated and described and that all changes and modifications that come within the spirit of the disclosure are desired to be protected.

There are a plurality of advantages of the present disclosure arising from the various features of the method, apparatus, and system described herein. It will be noted that alternative embodiments of the method, apparatus, and system of the present disclosure may not include all of the features described yet still benefit from at least some of the advantages of such features. Those of ordinary skill in the art may readily devise their own implementations of the method, apparatus, and system that incorporate one or more of the features of the present invention and fall within the spirit and scope of the present disclosure.

1. A heat and moisture exchange (HME) medical device, comprising:
an HME material configured to absorb heat and moisture from exhaled air and to return the heat and moisture to inhaled air;
a base including at least one wall defining a passageway for the exhaled and inhaled air;
a lid that retains the HME material, wherein the lid is movable between (i) a first position in which the lid is engaged with the base and the HME material is positioned in the passageway and (ii) a second position in which the lid is disengaged from the base and the HME material is not positioned in the passageway; and
a tether connecting the lid to the base both when the lid is in the first position and when the lid is in the second position.

2-12. (canceled)

13. The HME medical device of claim 1, wherein the tether provides a hinged connection between the lid and the base.

14. The HME medical device of preceding claim 1, wherein the tether comprises a living hinge.

15. (canceled)

16. The HME medical device of claim 1 wherein the lid is formed to include one or more openings that allow the exhaled and inhaled air to pass through the lid when the lid is in the first position.

17. The HME medical device of claim 16, wherein the lid further comprises a valve configured to selectively seal the one or more openings.

18. The HME medical device of claim 17, wherein at least a portion of the valve is moveable between (i) a closed position in which the portion covers the one or more openings and (ii) an open position in which the portion is spaced from the one or more openings, wherein the portion of the valve is biased toward the open position, and wherein the portion of the valve is configured to be moved from the open position to the closed position by a patient pressing a finger on the valve.

19-20. (canceled)

21. The HME medical device of claims 18, wherein a force between 0 and 1.7 newtons applied to the valve by the patient's finger is sufficient to seal the one or more openings.

22-25. (canceled)

26. The HME medical device of claim 1, wherein the HME material is removable from the lid and replaceable with another HME material.

27-28. (canceled)

29. The HME medical device of claim 1, wherein the lid includes one or more cross-bars that retain the HME material adjacent the lid.

30. (canceled)

31. The HME medical device of claim 29, wherein the cross-bars each extend from one side of the lid to an opposite side of the lid.

32-33. (canceled)

34. The HME medical device of claim 1, wherein the lid engages with the base in snap fit when the lid in the first position.

35. The HME medical device of claim 1, wherein the lid includes a first mating feature configured to engage a second mating feature of the base when the lid is in the first position.

36-37. (canceled)

38. The HME medical device of claim 1, wherein the base and the lid are configured to allow a patient to move the lid between the first and second positions with a single hand.

39. (canceled)

40. The HME medical device of claim 38, wherein a force between 0 and 9 newtons applied to the lid is sufficient to move the lid from the first position to the second position.

41-43. (canceled)

44. The HME medical device of claim 38, wherein a force of about 0.7 newtons applied to the lid is sufficient to move the lid from the first position to the second position.

45. The HME medical device of claim 1, wherein an air pressure between 0 and 100 centimeters of water in the passageway is sufficient to move the lid from the first position to the second position.

46-50. (canceled)

51. The HME medical device of claim 1, further comprising a tracheostomy tube defining an airway, wherein the base is configured to open into the airway.

52-55. (canceled)

56. The HME medical device of claim 1, wherein the base, the lid, and the tether are formed of molded polymer.

57-65. (canceled)

66. The HME medical device of claim 1, wherein the tether is separable from at least one of the base and the lid.

67. (canceled)

68. The HME medical device of claim 1, wherein the tether is mechanically connected to at least one of the base and the lid.

69-73. (canceled)
